(19) United States
(12) Patent Application Publication
(10) Pub. No.: US 2021/0025905 A1
(43) Pub. Date: Jan. 28, 2021

(54) Testosterone Saliva Test

(71) Applicant: Dawn Maslar, Ft Lauderdale, FL (US)

(72) Inventor: Dawn Maslar, Ft Lauderdale, FL (US)

(21) Appl. No.: 17/071,802

(22) Filed: Oct. 15, 2020

Related U.S. Application Data
(63) Continuation-in-part of application No. 16/151,284, filed on Oct. 3, 2018.
Application No. 16/151,284 is a continuation-in-part of application No. 15/833,702, filed on Dec. 6, 2017, now abandoned.

(51) Int. Cl. G01N 33/74

(52) U.S. Cl. CPC G01N 33/743

(57) ABSTRACT

A lateral flow assay format test that accepts a saliva sample that is reacted with a conjugate to indicate the level of testosterone present in the saliva sample.

### 1. RELATED APPLICATIONS

This application is a continuation-in-part of and claims benefit to U.S. non-provisional patent application Ser. No. 16/151,284, filed on 3 Oct. 2018 titled “Testosterone Saliva Test” and U.S. non-provisional patent application Ser. No. 15/833,702 filed on 6 Dec. 2017 titled “Testosterone Saliva Test”. Both are incorporated herein by reference in their entirety.

### II. BACKGROUND OF THE INVENTION

### 1. Field of the Invention

The present invention relates to human medical or fitness testing, and more particularly to a lateral flow assay format test strip having a simplified and visually verifiable threshold test to determined estimated blood testosterone levels.

### 2. Description of the Related Art

Several designs for testosterone testing in humans have been designed in the past. None of them, however, includes a portable and simple lateral flow assay test that can nearly instantly provide empirical results of a man's estimated testosterone level.

The present disclosure utilizes a lateral flow assay format with a control strip and a test strip that is used outside of laboratory-controlled conditions and having a simplified and visually verifiable threshold test to determined estimated blood testosterone levels by measuring free testosterone in saliva. Similarly, the present device employs a single strip with both the control and test markers.

Other publications and patents describing the closest subject matter provide for a number of more or less complicated features that fail to solve the problem in an efficient and economical way. None of these disclosures suggest the novel features of the present invention.

### III. SUMMARY OF THE INVENTION

It is one of the main objects of the present invention to provide a simple and efficient device to test human testosterone levels derived from a saliva sample.

It is still another object of the present invention to provide an accurate test to determine human testosterone levels using a calibrated test solution run in parallel to a saliva test. It should be appreciated that a single run could also include both the test and control features.

It is yet another object of this invention to provide such a device and method of use that is inexpensive to manufacture and maintain while retaining its effectiveness.

Further objects of the invention will be brought out in the following part of the specification, wherein detailed description is for the purpose of fully disclosing the invention without placing limitations thereon.

### IV. BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 shows a perspective view of an example of a testosterone test.

FIG. 2 shows a plan view of a testosterone saliva test.

FIG. 3 shows a plan view of a reader assembly internal components.

FIG. 4 shows an exploded perspective view of a cassette assembly.

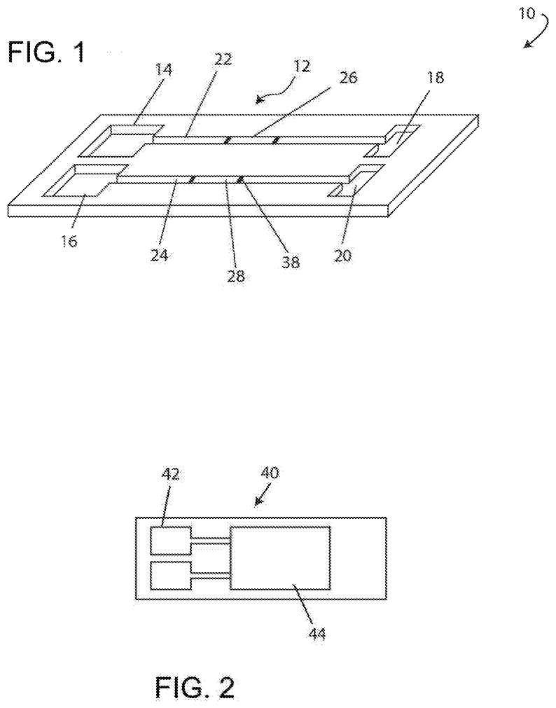

### V. DETAILED DESCRIPTION OF THE EXEMPLARY EMBODIMENTS

The subject device and method of use is sometimes referred to as the device, the invention, the test, the testosterone test, the saliva test, the machine or other similar terms. These terms may be used interchangeably as context requires and from use, the intent becomes apparent. The masculine can sometimes refer to the feminine and neuter and vice versa. The plural may include the singular and singular the plural as appropriate from a fair and reasonable interpretation in the situation.

The purpose of this test is to quantify testosterone levels in human saliva for purposes of health, athletics, relationships or personal information.

An exemplary embodiment of the present invention has chromatographic separation, but we can now use a different technique than what the prior art describes. Chromatography is the separation of dissolved substances by interaction with a material. There are dozens of different types of chromatographic techniques involving a stationary phase and a mobile phase, including column chromatography, gas chromatography, thin-layer chromatography and ion chromatography, to name a few.

The present process is a chromatographic competition method and not a chromatographic separation, as has been known in the prior art. Instead, it is a type of fractional technique that may be referred to affinity chromatography.

Some prior art techniques teach that when the liquid containing the suspended microparticles arrives at the region of the chromatographic strip bearing the second binding material, the analyte (if present in the original sample) will form a molecular bridge between the analyte-binding material on the microparticles and the analyte-binding material affixed to the strip, resulting in the immobilization of the microparticles at that point on the strip where the analyte-binding material is affixed. This immobilization of the microparticles results in a visible signal (e.g., a colored band or dot) at this point on the strip. If the analyte is not present in the sample, the microparticles will continue past the location on the chromatographic strip and a visible signal will not be produced. This describes a sandwich assay where the fluid moves along the strip and if the desire analyte is present, it is separated out at the reactant line causing a color change.

Prior art also teaches that if the analyte is present in the test sample, it will combine with its labeled binding partner to form a complex which will flow along the strip to a detection zone which contains a substrate for the enzyme label capable of providing a signal (e.g., a colored response) in the presence of the enzyme label. This is an accepted definition of a sandwich assay.

The present test is different. Applicant's test is a competitive lateral flow assay to precisely and quantitatively determine the testosterone levels in the saliva derived from the comparative difference between the darkness of the test line and the control line. These should be precisely measured. Comparing these two lines is by definition a competitive assay, which is different from a sandwich assay where the control line is independent. An aspect of the present solution is to look to see the change to the test and the control area to measure the change in these areas after exposure to the saliva, in order to determine the concentration of testosterone in the saliva with a high degree of quantitative accuracy.

In the present method, the saliva fluid does move down the strip, however, a blocker competes with our desired analyte. Our analyte is not simply separated out, but instead it chromatographically competes for the affinity on the same location. If the blocker is bound then the analyte can't bind. Instead of a more intense line with the presence of the analyte, as seen in the sandwich assays found in the prior art, a reduced signal with higher testosterone and a fainter test line is made. That is why we have to use a unique calculation of comparing the test line to the control line (test/control) to be able to calculate the quantity. It's not simply the intensity of the test line. Therefore, the present design employs a separation technique that is very different from the prior art.

An electronic sensor is paired with the test body that optically determines an intensity of the test visual indicator relative to an intensity of the control visual indicator then calculates and displays in a human readable format a quantified concentration of testosterone in a saliva sample.

This calculation is comparing the two lines (test and control) regardless of background. In the present test the background could be a variety of colors but would not have an effect on the test, because it is comparing lines that are so close together on the same strip of paper that it may be assumed the background is the same. This is different from the prior art. The prior art compares the test line against a background of blank white paper.

The closest prior art shows that with the use of a background with the total fluorescence on the assay sample the line is determined, and a measurement of “background” fluorescence is subtracted from the total fluorescence. In another exemplary embodiment, the total fluorescence on the control sample line is determined, and a measurement of “background” fluorescence from the region of the assay that shouldn't have fluorescence is subtracted from the total fluorescence.

In the present test, a comparison is made between the test line with the control line. There is no comparison to “background” as reported by the prior art where the background is subtracted. This is found in that fluorophore may be used. This reagent requires the “background” subtraction which is not required with other reagents since it's based on the excitation state across the visual light spectrum and they are prone to bleed-through emission.

The present design does not use fluorophore or any type of reagent that has a bleed through. However, even it did, the bleed through is irrelevant in this case because it's not the surrounding paper that's important. Rather is the relative darkness of the test and control line compared to each other.

Referring now to the drawings, where the present invention is generally referred to with numeral 10, it can be observed that it basically includes a kit body 12, a well or pad 14, a well or pad 16, a pad 18, a pad 20, a strip 22, a strip 24, a conjugate 26 and a conjugate 28.

Generally, the kit body 12 is fabricated of a rigid and durable material. A plastic kit body 12 approximately 4 cm deep, 12 cm wide and 1 cm in height can be effective. These materials and dimensions are not intended to be limited, but merely a practical example of rough dimensions to aid the reader in understanding the scale of the device. Clearly, larger, smaller or differing materials of the kit body 12 will be effective and fall within the inventive scope of the device.

Formed into the kit body 12 is strip 22 that is substantially parallel to strip 24 and span the width of the device. A well or pad 14 is placed on one end of the strip 24 and is dimensioned and adapted to accept a saliva sample. The pad may also stick out separately from the device. Similarly, a well or pad 16 is located at one end of strip 24 and is dimensioned and adapted to receive a control sample specimen. The wells 14 and 16 are similarly dimensioned so they perform similarly. The strips 22 and 24 are also similarly dimensioned and positioned so they perform nearly the same as each other. An absorption pad, osmotic device, capillary action device or other saliva collection and delivery may be used alternately than the well 14 and strip 24.

When a sample of saliva is placed into well or pad 14, it wicks along the strip 22. The saliva wicks through and past the conjugate 26 zone where the saliva reacts with a compound incorporated into the strip 22. The compound may be gold nanoparticles with antibodies or antigens that react in the presence of testosterone. Other chemistries of conjugate 26 can also be used depending on availability, sensitivity, cost and effectiveness as detecting trace amounts of testosterone in the saliva of the test subject.

At the other end of the strip 22 is a pad 18. The strip 24 also has a pad 20 opposite the well 16 end of the strip 24. The pads 18 and 20 absorb saliva and the control solution to ensure that the tested samples flow along the length of the respective strip 22 and strip 24. This ensures that reliably equal volumes of liquid tested will pass through the conjugates 26 and conjugate 28. The absorbent nature of the pads 18 and 20 help prevent overflow and spilling of tested fluids.

Similar to the saliva, a control test solution is placed into the well or pad 16 as part of the test. The control solution wicks along the strip 24 through the conjugate 28 in the central section of the strip 24 as the control solution is drawn by wicking towards the pad 20 where any excess solution is absorbed. There may also be two wells that perform in parallel, each with a strip.

The control solution has a precisely calibrated concentration of testosterone or other appropriate analog of testosterone. The concentration of testosterone in the control solution should be equal to the testosterone found in saliva when a test subject would have about 360 pMol/L of testosterone in their blood. This test/device could be used for differing levels of concentrations that the user is desirous of measuring.

To aid a user in reading the results of the test there may be one or more indicators similar to indicator 38. The indicator 38 may be a simple line or pair of lines that are revealed when a threshold concentration of testosterone is wicked through the conjugate 28 zone. If multiple indicators 38 are present it is possible to have different concentrations activate adjacent indicators 38. For example, a series of indicators 38 may be progressively along the strip 24 with one revealing itself at a first concentration (i.e. 300 pMol/L testosterone), the next indicating with a higher ratio (i.e. 360 pMol/L) and a third indicator turns darker or lighter only when presented with a higher ration of testosterone in the wicked fluid (i.e. 450 pMol/L). In this fashion, a sample can be determined to be in a particular range. Of course, the specific range points of the indicators 38 can be made to any desired concentration as would be appropriate for the specific test parameters. In at least one specific chemistry lightening of the indicator 38 progressively as higher concentrations of testosterone is found has been effective.

The control solution should be precisely calibrated to a specific concentration. For example, in the representative ratios above, the control solution could be precisely 360 pMol/L. An application of the control solution to the well 16 would wick the solution past the three indicators 38 and the first two would cause an indication but the 450 pMol/L indicator would not change because that concentration was not achieved in the test. Again, these are mere examples for a better understanding of a typical type of device and the specific numbers of indicators 38 and concentrations they alert at can be adjusted.

In another exemplary embodiment, an indicator 38 may darken or indicate when it is moistened by the test fluid or saliva regardless of testosterone concentration and the second indicator 38 may be set to indicate a specific level of testosterone detected. In this example, the first indicator 38 the saliva or test fluid meets while wicking along the well 24 could be the one calibrated to alert at a specific testosterone concentration and the second indicator 38, nearer the pad 20, can alert on any moisture thereby proving that a sample of sufficient volume was put into the well to initiate the testing.

Generally, both sides of wells and pads 14 and 16, pads 18 and 20, strips 22 and 24, conjugates 26 and 28 and any indicators 38 present will be the same for both sides of the kit body 12 so that either side can be equally used by the control solution or saliva interchangeably. In some cases the control solution (sometimes referred to as the test solution) will behave differently than the saliva so that each side of the test body 12 will not be identical but will have chemical differences to get the achieved results of reliably checking the control against the saliva sample in order to ensure repeatable and accurate results.

Accessories that may be included in a test kit to include, among other things and features, a pipette 30 and a cup 32. Depending on the saliva sample size, the size of the cup, the individual user or other factors, it may be necessary to expectorate saliva into a cup 32 instead of directly into the well 14. Also, a reagent may be added to the saliva. The reagent may help with decreasing viscosity and/or enhance the sample for testing

A pipette 30 (or dropper, etc.) may also be provided with the cup to remove a precise volume of control solution or saliva into the wells 14 and 16. The use of a pipette 30 can also help avoid sampling of saliva with foam or other undesired parts. The pipette 30 can be precisely calibrated to pull a precise volume of fluid by capillary action to be dispensed into the wells 14 and 16 for the test. In another exemplary embodiment, the saliva sample may be extracted from the mouth by an absorbent material.

FIG. 2 shows some alternative and optional features on variations of the inventive concept and include, among other features, a kit body 40, a well 42 and an indicator 44. Any of these features and elements can be interchanged with other discussed elements to achieve the results of accurate saliva testing for testosterone.

The present invention involves a test based on the detection of male salivary testosterone reaching a threshold level using lateral flow immunoassay.

Testosterone is a steroid hormone produced by men and women. Levels are generally naturally higher in men. Normal levels can range from about 260-1250 ng/dL.

Currently, this type of detection would require an enzyme-linked immunosorbent assay (ELISA) immunoassay. This can require several days and significant cost. For an ELISA test, a sample is typically sent to a lab using specialized instruments and highly trained personnel to operate the equipment.

Current rapid-result testosterone tests require a blood sample, which makes participation in the testing less appealing. Blood samples may also require a nurse or other trained technician to draw the blood sample and conduct the test. This is a less private situation than couples may want to undertake. A test is needed that can be performed without significant training and skill in the home of office of people taking and administering the test.

In order to overcome these drawbacks, the present test and method of testing provides a quantitative near-instant assay and detection of salivary testosterone utilizing lateral flow immunoassay. This can manifest, for example, in a competitive assay format, Monoclonal [DM1] antibodies may be utilized that are similar to those used in an ELIZA test.

In one exemplary embodiment, the test strip is comprised of a membrane strip, a sample pad, a conjugate (detector) pad, an absorbent (sink) pad and antibody and antigen reagents.

The sample may be provided by the test subject by spitting into a well on the kit body. The well's design may ensure a quantifiable amount of saliva will be deposited on the pad if needed. In addition to the sample amount, another control fluid may be added and will run parallel to the sample amount. If a control sample is used, the control sample will contain a known amount of testosterone or testosterone analog to use as a comparison to the saliva sample from the test subject. A package may be provided that includes the test body, instructions and a vial of control sample for convenient testing wherever and whenever needed.

Continuing this example, both the well and the control will be in fluid communication with the membrane strip that is part of the lateral flow assay method and device. Basic components of lateral flow assay test bodies are commercially available and consist generally of a sample pad with a pretreatment buffer that may contain blockers to prevent errors from interference. The sample pad is separated from at nitrocellulose membrane by the conjugate pad. The conjugate may consist of latex beads, gold nanoparticles or colored reagent combined with antibodies or antigens. The amount of detector molecules is specifically calibrated in this test to measure the amount of testosterone desired. The antibody or antigen may be conjugated with a colorant such as latex beads, dyes or nanoparticles.

The test body configuration would lead the saliva sample to a membrane, for example a nitrocellulose membrane, that would wick into either a color reaction or a signal that a digital reader can read. This membrane is then compared to the results achieved by the control. to verify the testosterone threshold for a positive result is met. If not, the test or reagents may be faulty and a new test should be considered to gain results within the predetermined margin of error for that test as determined during manufacturing.

The final piece would consist of an absorbent pad to prevent back flow of the liquid saliva or test (calibration) liquid from draining out of the test body. This can improve safety for contact with body fluids (saliva) and generally make a cleaner test procedure.

A related exemplary embodiment is to create a thermometer or tube-like device or absorbent pad that would wick the saliva directly from a man's mouth using capillary action. When the saliva reaches the top, it interacts chemically with an indicator compound and would signal a positive or negative result, as appropriate. The test taker may then remove the apparatus, and place a cap over the tip. The cap would ensure applied pressure to release the sample from the capillary and release the control onto the sample pad.

In another exemplary embodiment of the testosterone saliva test, a single strip, well, pad and conjugate are used instead of requiring a pair having one for the saliva and the other for the control fluid. In this version, only the saliva is used for the test. No verification of the accuracy of the test is required by use of a control fluid.

To control the flow of saliva and normalize the assay, the saliva receiving pad (or other analogous component described herein) may include a carrying fluid or pushing fluid that may combine with the saliva sample to effectively travel or flow in concert along the path of the assay over the test markers.

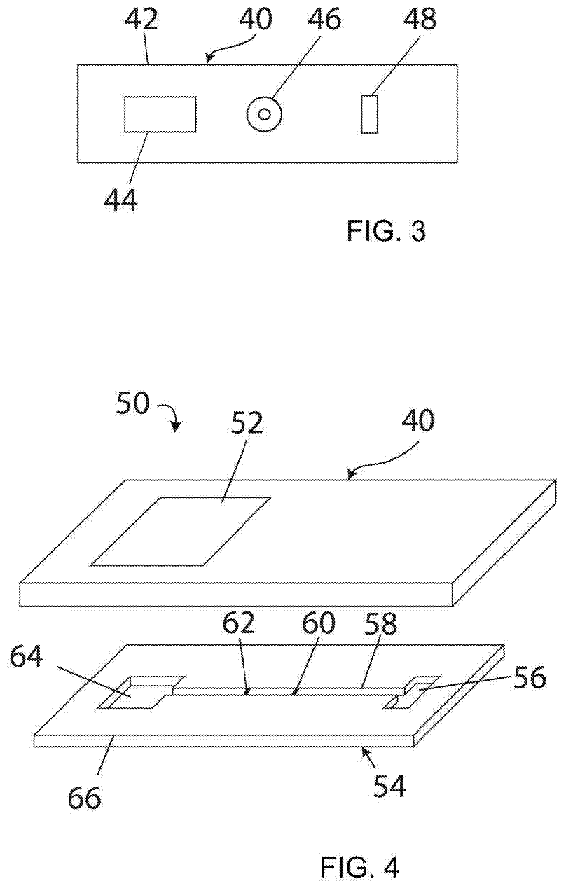

FIGS. 3 and 4 show an exemplary embodiment of the test that also includes an electronic optical reader in combination with the lateral flow assay test to help with more precise and optionally quantitative results of testosterone concentration in the saliva sample. The cassette assembly 50 example includes, among other features, a reader assembly 40, a body 42, a processor 44, a camera 46, a battery 48, a display 52, an assay assembly 54, an absorption pad 56, a path 58, a test indicator 60, a control indicator 62, a saliva pad 64 and a tray 66.

The reader assembly 40 is a compact electronic device that is adapted to fit onto an assay assembly 54 combining to form the cassette assembly 50. The reader assembly 40 is powered by a battery 48 and controlled by a processor 44. The processor 44 and battery 48 work together with an integrated camera 46 on the interior surface. An electronic display 52 is on the exterior surface of the reader assembly 40.

The assay assembly 54 is similar to any of the versions disclosed herein. A saliva sample is placed onto the saliva pad 64 and travels along the path 58 towards the absorption pad 56. As the saliva moves over the control line 62 and test line 60, or the lines may be reversed. The reagents will react to predetermined degrees of dark or light depending on the concentration of testosterone in the saliva sample.

For some testosterone reagent combinations, the absolute darkness of the test line 60 and control line 62 and relative darkness of the test line 60 and control line 62 are difficult to precisely determined with the human eye. To precisely and quantitatively determine the testosterone levels in the saliva the darkness of the test line 60 and control line 62 should be precisely measured.

To precisely measure the darkness of the test line 60 and control line 62 the reader assembly 40 is fit over the assay assembly 54 so that the camera 46 is positioned to optically view the test line 60 and control line 62. The tray 66 may be provided attached to the body 42 in one compact unit that protects the electronics of the reader assembly 40 and the reagents in the assay assembly 54.

The reader assembly 40 is turned on with a switch or is activated by the presence of the saliva sample on the saliva pad 64. The camera 46, controlled by the processor 44, reads the darkness of the test line 60 and control line 62. The darkness value of the control line 62 may be established as a datum for relative darkness of the test line 60. An empirical value for the darkness of the saliva reacted test line 60 may therefore be established. The darkness value may be a specific number value of darkness that may then be displayed on the display 52.

The test includes a lateral flow assay test style test integrated into a test body, for example, a small plastic slab. The test body is adapted to accept a saliva sample that is drawn into the active areas of the test body. The test body has adhered to a test area a testosterone reactive conjugate. The saliva flows to the test area during the initial phases of the test. The test body has a control visual indicator that when reacted with the saliva sample proves visually that the testosterone reactive conjugate is reacting effectively with testosterone in the saliva sample. This could be a line appearing in the test area or other positive indicator. The test body has a test visual indicator that when reacted with the saliva sample visually indicates the concentration of testosterone in the saliva sample by varying intensity of the test visual indicator. This can be a lighter or darker line depending on the chemistry of the reagents. Optionally, an electronic sensor is paired with the test body that optically determines an intensity of the test visual indicator relative to an intensity of the control visual indicator then calculates and displays in a human readable format a quantified concentration of testosterone in the saliva sample. This is essentially a camera that sees the changes to the test and control areas to measure the change in these areas after exposure to the saliva to determine the concentration of testosterone in the saliva with a high degree of quantitative accuracy.

An important version of the invention can be fairly described as an affinity chromatography competition test for testosterone comprised of a competitive lateral flow assay test integrated into a test body. The test body is configured to accept a saliva sample and in at least one version is generally only a male sample. The test body has adhered to a test area a testosterone reactive conjugate over a background of the test body. Delivering the accepted saliva sample to both the testosterone reactive conjugate and a test visual indicator without any chromatographic separation of the saliva being required. The test body has a control visual indicator that when reacted with the saliva sample proves visually that the testosterone reactive conjugate is reacting effectively with testosterone in the saliva sample. The test body has a test visual indicator that when reacted with the saliva sample visually indicates the concentration of testosterone in the saliva sample by comparing a first intensity of the test visual indicator to a second intensity of the control visual indicator without respect to the background by determining the concentration of a component of saliva with the visual darkness of a line, without the use of a background to further determine the amount of analyte. Deriving a testosterone quantity in the saliva sample by comparing the first intensity with the second intensity. Optionally, an electronic sensor is paired with the test body wherein the test body optically determines an intensity of the test visual indicator relative to an intensity of the control visual indicator and then calculates and displays in a human readable format a quantified concentration of testosterone in the saliva sample.

The foregoing description conveys the best understanding of the objectives and advantages of the present invention. Different exemplary embodiments may be made of the inventive concept of this invention. It is to be understood that all matter disclosed herein is to be interpreted merely as illustrative, and not in a limiting sense. The invention is therefore only limited by the appended claims.

I claim:

1. An affinity chromatography competition test for testosterone comprised of:
a competitive lateral flow assay test integrated into a test body;
the test body is configured to accept a male only saliva sample;
the test body has adhered to a test area a testosterone reactive conjugate over a background of the test body;
delivering the accepted saliva sample to both the testosterone reactive conjugate and a test visual indicator without any chromatographic separation of the saliva being required;
the test body has a control visual indicator that when reacted with the saliva sample proves visually that the testosterone reactive conjugate is reacting effectively with testosterone in the saliva sample;
the test body has a test visual indicator that when reacted with the saliva sample visually indicates the concentration of testosterone in the saliva sample by comparing a first intensity of the test visual indicator to a second intensity of the control visual indicator without respect to the background by determining the concentration of a component of saliva with the visual darkness of a line, without the use of a background to further determine the amount of analyte;
deriving a testosterone quantity in the saliva sample by comparing the first intensity with the second intensity.

2. The affinity chromatography competition test for testosterone as in claim 1, further characterized in that an electronic sensor is paired with the test body;
wherein the test body optically determines an intensity of the test visual indicator relative to an intensity of the control visual indicator and then calculates and displays in a human readable format a quantified concentration of testosterone in the saliva sample.
